(19) United States
(12) Patent Application Publication
(10) Pub. No.: US 2022/0323015 A1
(43) Pub. Date: Oct. 13, 2022

(54) MEASUREMENT APPARATUS AND MEASUREMENT METHOD

(71) Applicant: SHARP KABUSHIKI KAISHA, Sakai City, Osaka (JP)

(72) Inventors: RYOTA TOMIZAWA, Sakai City, Osaka (JP); YOSHIHISA ADACHI, Sakai City, Osaka (JP); YOSHIFUMI IWAI, Sakai City, Osaka (JP); YUKI EDO, Sakai City, Osaka (JP); RIEKO OGAWA, Sakai City, Osaka (JP)

(21) Appl. No.: 17/634,384

(22) PCT Filed: Jun. 26, 2020
(86) PCT No.: PCT/JP2020/025114

§ 371 Date: Feb. 10, 2022

(30) Foreign Application Priority Data
Sep. 6, 2019 (JP) 2019-162539

(51) Int. Cl. A61B 5/00; A61B 5/024; A61B 5/021

(52) U.S. Cl. CPC A61B 5/7203; A61B 5/024; A61B 5/02125; A61B 5/7278

(57) ABSTRACT

A measurement apparatus comprises a determining unit and a correcting unit. The determining unit repeats selecting two pieces of waveform data from among three or more pieces of waveform data including attribute values and observation values corresponding to the attribute values and determining a difference between the attribute values in the selected two pieces of waveform data and a correlation in shape between the selected two pieces of waveform data, to determine difference-correlation pairs, each being a pair of the attribute value difference and the correlation. The correcting unit that corrects the attribute value difference, based on the correlation paired with the attribute value difference.

### TECHNICAL FIELD

The present disclosure relates to a measurement apparatus and a measurement method. This application claims priority to Japanese Patent Application No. 2019-162539 filed on Sep. 6, 2019, the entire contents of which are herein incorporated by reference.

### BACKGROUND ART

Conventionally, a technology for detecting pulse waves, for example, by referring to a moving image acquired by photographing a living body, such as a human body, has been known (for example, see PTL 1). PTL 1 describes one example of a living-body information obtaining apparatus that derives living-body information from a moving image obtained by image-capturing a living body.

The living-body information obtaining apparatus described in PTL 1 comprises region identifying means, pulse-wave detecting means, and phase-difference determining means. By performing image processing, the region identifying means identifies regions corresponding to respective two portions of a living body in frame images that constitute a moving image. The pulse-wave detecting means refers to the regions, identified by the region identifying means, to detect pulse waves of the respective two portions. The phase-difference determining means determines a phase difference between the pulse waves of the two portions, the pulse waves being detected by the pulse-wave detecting means. Various types of living-body information can be determined using the phase difference between the pulse waves.

### CITATION LIST

### Patent Literature

PTL 1: International Publication No. 2015/045554

### SUMMARY OF INVENTION

### Technical Problem

In the living-body information obtaining apparatus described in PTL 1, there are demands for enhancing the detection accuracy of pulse waves in order to accurately determine various types of living-body information.

A primary object of the present disclosure is to improve the measurement accuracy of a measurement apparatus.

### Solution to Problem

A measurement apparatus according to one aspect of the present invention comprises a determining unit and a correcting unit. The determining unit repeats selecting two pieces of waveform data from among three or more pieces of waveform data including attribute values and observation values corresponding to the attribute values and determining a difference between the attribute values in the selected two pieces of waveform data and a correlation in shape between the selected two pieces of waveform data, to determine difference-correlation pairs, each being a pair of the attribute value difference and the correlation. The correcting unit that corrects the attribute value difference, based on the correlation paired with the attribute value difference.

In a measurement method according to one aspect of the present invention, selecting two pieces of waveform data from among three or more pieces of waveform data including attribute values and observation values corresponding to the attribute values and determining a difference between the attribute values in the selected two pieces of waveform data and a correlation in shape between the selected two pieces of waveform data are repeated to determine difference-correlation pairs, each being a pair of the attribute value difference and the correlation; and the attribute value difference is corrected based on the correlation paired with the attribute value difference.

### BRIEF DESCRIPTION OF DRAWINGS

FIG. 1 is a block diagram of a measurement apparatus.

FIG. 2 is a flowchart showing a flow of processes for determining pulse wave propagation times.

FIG. 3 is a diagram showing relationships of pulse-wave data f₁, f₂, and f₃ in a first embodiment.

FIG. 4 is a graph schematically showing the pulse-wave data f₁ and f₂.

FIG. 5 is a graph schematically showing a function R₂₁(τ).

FIG. 6 is a graph showing relationships of pulse-wave data f₁, f₂, f₃, f₄, and f₅ in a third embodiment.

### DESCRIPTION OF EMBODIMENTS

One example of preferable modes in which the present invention is implemented will be described below. Embodiments described below, however, are merely exemplary. The present invention is not by no means limited to the embodiments described below.

### First Embodiment

(Measurement Apparatus 1)

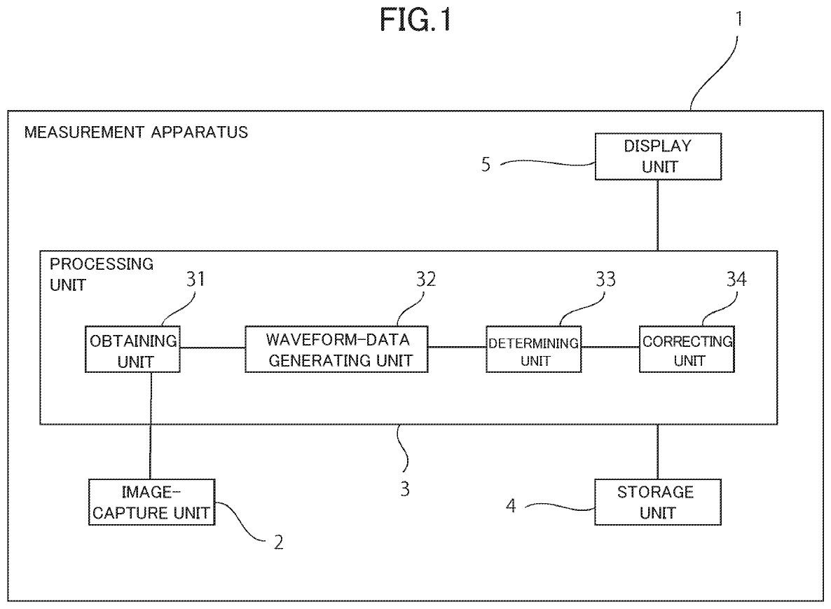

FIG. 1 is a block diagram of a measurement apparatus 1 according to a first embodiment. The measurement apparatus 1 shown in FIG. 1 is, for example, a measurement apparatus that can measure living-body information of a person to be measured. The living-body information that can be measured by the measurement apparatus 1 is, for example, waveform data, such as a pulse wave, that can be obtained from a living body or data that can be determined using the waveform data. Specific examples of the data that can be determined using the waveform data include, for example, a pulse wave propagation time, a pulse rate, a respiration rate, and so on.

Specifically, by using two pieces of waveform data including attribute values and observation values corresponding to the attribute values, the measurement apparatus 1 determines a difference between the attribute values in the two pieces of waveform data and outputs the difference. Herein, the “observation value” in the waveform data is the amount of change in the waveform data and refers to a physical quantity to be measured. For example, a pressure, a volume, a temperature, or the like is exemplified as the physical quantity to be measured. The physical quantity to be measured may be, for example, a physical quantity to be measured itself or may be a value that is correlated with a physical quantity to be measured and that is output from a measuring instrument. For example, when the physical quantity to be measured is a pressure, and a sensor that detects a pressure and that outputs a voltage having a level corresponding to the detected pressure is used, the observation value may be, for example, a pressure value or a voltage value that is output.

The “attribute value” in the waveform data is a value representing an attribute of the observation value. For example, when the waveform data represents changes over time in the observation value, the attribute value means a time point at which an observation value corresponding to the attribute value is observed. For example, when the waveform data indicates changes in the observation value in space, the attribute value means a position where an observation value corresponding to the attribute value is observed.

Specifically, the measurement apparatus 1 in the present embodiment is an apparatus that measures a pulse wave propagation time, which is a difference between attribute values, by using pulse-wave data, which is waveform data in which the attribute values are time points.

The “pulse wave” is a wave obtained by representing blood vessel pulsation associated with blood ejection of the heart as a waveform. Thus, the pulse-wave data is waveform data indicating changes over time in the observation value at an arbitrary spot. The pulse wave includes a pressure pulse wave and a volume pulse wave.

The “pressure pulse wave” is a wave obtained by representing blood vessel pulsation associated with blood ejection of the heart as a waveform.

The “volume pulse wave” is a pulse wave obtained by representing changes in the volume of a blood vessel as a waveform.

A “pulse wave propagation speed” refers to a speed at which a pulse wave propagates in a blood vessel. The pulse wave propagation speed can be determined, for example, by dividing the length of a blood vessel between two portions of a living body by a phase difference (a lag in arrival time) between pulse waves at the two portions.

The “pulse wave propagation time” is a time taken for a pulse wave to propagate from one portion of the living body to a portion different from that portion. The length of the blood vessel between the two portions from which the pulse wave propagation time is measured can be determined, for example, by multiplying the pulse wave propagation time and the pulse wave propagation speed.

As shown in FIG. 1, the measurement apparatus 1 comprises an image-capture unit 2, a processing unit 3, a storage unit 4, and a display unit 5.

The image-capture unit 2 captures an image (in the present embodiment, specifically, a moving image), which is data for generating pulse-wave data that is waveform data. The image-capture unit 2 has, for example, an image-capture element, such as a CCD (charge coupled device), a CMOS (complementary metal-oxide semiconductor device), or the like. The image-capture unit 2 may have one image-capture element or may have a plurality of image-capture elements.

The image (moving image) may be an image in any wavelength range. The image (moving image) may be, for example, an image in a visible wavelength range, an image in a near-infrared range, or an image in an infrared range.

The image-capture unit 2 image-captures a subject (a person to be measured) to generate a moving image. The image-capture unit 2 outputs the generated moving image to the processing unit 3. When the image-capture unit 2 has a plurality of image-capture elements, the image-capture unit 2 may merge moving images captured by the respective image-capture elements and output them as one moving image.

The image-capture unit 2 image-captures the subject for a pre-set image-capture period (for example, for 30 seconds). After finishing the image capturing, the image-capture unit 2 may output a moving image to the processing unit 3 at a time, or during the image capturing or after the image capturing, the image-capture unit 2 may divide a moving image and output resulting moving images to the processing unit 3 at a plurality of times.

The image-capture unit 2 may have, for example, a storage unit, in addition to the image-capture element. In this case, the image-capture unit 2 may temporarily store the moving image, captured by the image-capture element, in the storage unit and output the moving image, stored in the storage unit, to the processing unit 3.

The processing unit 3 determines a pulse wave propagation time by using the moving image input from the image-capture unit 2. Specifically, the processing unit 3 generates three or more pieces of waveform data (pulse-wave data) by using the moving image. The processing unit 3 selects two pieces of waveform data from among the three or more pieces of waveform data and determines a pulse wave propagation time, which is a difference between attribute values (time points), by using the selected two pieces of waveform data. The processing unit 3 can be constituted by, for example, a central processing unit (CPU).

The storage unit 4 is connected to the processing unit 3. The storage unit 4 stores information output from the processing unit 3. Also, the storage unit 4 outputs the information stored in the storage unit 4 to the processing unit 3, in response to an instruction from the processing unit 3. The storage unit 4 can be constituted by, for example, a RAM (random access memory).

The display unit 5 is connected to the processing unit 3. The display unit 5 displays, for example, the pulse wave propagation time determined by the processing unit 3, the waveform data generated by the processing unit 3, or the like in response to an instruction from the processing unit 3. The display unit 5 can be constituted by, for example, various types of display panel, such as a liquid-crystal display panel.

The measurement apparatus 1 may further comprise, for example, an output unit, such as a printer or a plotter, for outputting the determined pulse wave propagation time, the generated waveform data, or the like.

Next, a configuration of the processing unit 3 will be described in detail. The processing unit 3 has an obtaining unit 31, a waveform-data generating unit 32, a determining unit 33, and a correcting unit 34.

The obtaining unit 31 obtains, from the image-capture unit 2, an image (specifically, a moving image) as information for generating waveform data. The obtaining unit 31 outputs the obtained moving image to the waveform-data generating unit 32.

The waveform-data generating unit 32 generates three or more pieces of waveform data (pulse-wave data) from the input moving image. Specifically, the waveform-data generating unit 32 generates pulse-wave data of three or more different portions of the subject. Specifically, the waveform-data generating unit 32 selects three or more portions from a region where skin of a person to be measured who is a subject is image-captured. The three or more portions to be selected are, generally, portions whose distances from the heart are different from each other. The waveform-data generating unit 32 generates pulse-wave data of the portions selected from the moving image.

A method for generating the pulse-wave data from the moving image is not particularly limited. The waveform-data generating unit 32 may generate the pulse-wave data from the moving image, for example, by applying an independent component analysis method or a color component separation method.

Specifically, the waveform-data generating unit 32 generates the pulse-wave data, for example, by using the intensity of a color (for example, green (G)) having a predetermined tone at each portion as observation values.

The above-described method for generating the pulse-wave data is a method that pays attention to a property that hemoglobin included in blood absorbs light in particular color (for example, green). The waveform-data generating unit 32, which generates pulse-wave data by using the method for generating the pulse-wave data, detects time-series changes in color of the surface of skin at each portion, the changes being caused by blood flow, from a moving image and generates pulse-wave data (volume pulse-wave data) from the detected time-series changes in the color in an approximate manner.

The number of pieces of waveform data generated by the waveform-data generating unit 32 is not particularly limited as long as the number thereof is three or more.

The determining unit 33 repeats selecting two pieces of waveform data from among three or more pieces of waveform data (pulse-wave data) and determining a difference (specifically, a pulse wave propagation time) between the attribute values in the selected two pieces of waveform data and a correlation in shape between the selected two pieces of waveform data. As a result, the determining unit 33 determines a plurality of difference-correlation pairs (pairs (pulse wave propagation times, correlations)), each being a pair of the attribute value difference (specifically, a pulse wave propagation time) and the difference. The determination of the plurality of pairs (pulse wave propagation times, correlations), the determination being performed by the determining unit 33, are detailed later.

The correcting unit 34 corrects the pulse wave propagation time, which is the difference between the attribute values measured by the determining unit 33, based on the correlation paired with the difference (the pulse wave propagation time) between the attribute values. Specifically, the correcting unit 34 corrects at least one of the pulse wave propagation times measured by the determining unit 33, based on the correlation paired with the at least one pulse wave propagation time. The correction of the pulse wave propagation time in the correcting unit 34 is detailed later.

(Measurement Method for Pulse Wave Propagation Times)

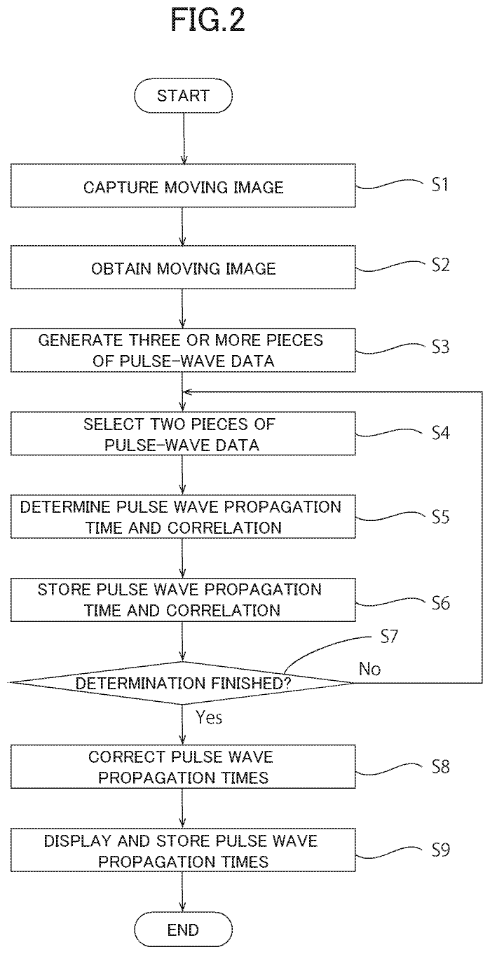

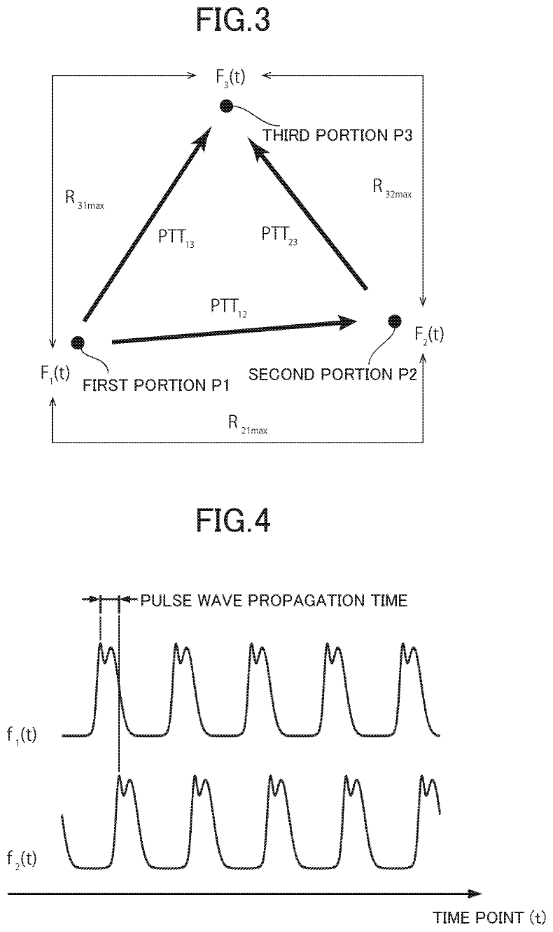

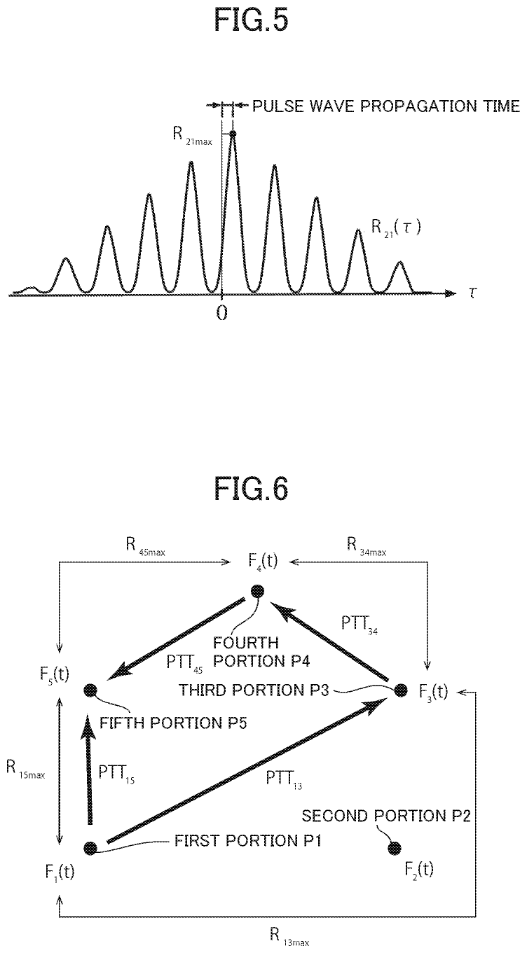

FIG. 2 is a flowchart showing a flow of processes for determining pulse wave propagation times in the first embodiment. FIG. 3 is a diagram showing relationships of pulse-wave data f₁, f₂, and f₃ in the first embodiment. FIG. 4 is a graph schematically showing the pulse-wave data f₁ and f₂. FIG. 5 is a graph schematically showing a function R₂₁(τ). Next, a measurement method for pulse wave propagation times, each being a difference between attribute values, by using the measurement apparatus 1 will be described in detail with reference to FIGS. 2 to 5.

(Measurement Method for Pulse Wave Propagation Times)

As shown in FIG. 2, first, the image-capture unit 2 image-captures a subject (a person to be measured) to generate a moving image (step S1). Specifically, the image-capture unit 2 generates a moving image of skin exposed from clothing of the person to be measured. The image-capture unit 2 outputs the captured moving image to the obtaining unit 31 in the processing unit 3.

Next, the obtaining unit 31 obtains the moving image, which is information for generating pulse-wave data that is waveform data (step S2). The obtaining unit 31 outputs the obtained moving image to the waveform-data generating unit 32. The obtaining unit 31 may cause the obtained moving image to be stored in the storage unit 4. In this case, the waveform-data generating unit 32 may be adapted to access the storage unit 4 to read the moving image stored in the storage unit 4.

Next, the waveform-data generating unit 32 generates three or more pieces of waveform data (pulse-wave data) from at least one moving image (step S3). Specifically, by using the moving image, the waveform-data generating unit 32 generates pulse-wave data for three or more different portions of the person to be measured. For example, the waveform-data generating unit 32 detects time-series changes in color of the surface of skin at each portion from the moving image and generates pulse-wave data from the detected time-series changes in the color. An example in which the waveform-data generating unit 32 generates pulse-wave data for three portions, namely, a first portion P1, a second portion P2, and a third portion P3 (see FIG. 3), will be described below in the present embodiment.

The waveform-data generating unit 32 outputs the generated pulse-wave data to the determining unit 33. The waveform-data generating unit 32 may store the generated pulse-wave data in the storage unit 4. In this case, the determining unit 33 may be adapted to access the storage unit 4 to read the pulse-wave data stored in the storage unit 4.

The determining unit 33 selects two pieces of pulse-wave data from among the three or more pieces of pulse-wave data (step S4). Next, the determining unit 33 determines a pulse wave propagation time, which is a difference between the attribute values in the two pieces of waveform data selected in step S4, and a correlation in shape between the selected two pieces of pulse-wave data (step S5). Next, the determining unit 33 stores the determined pulse wave propagation time and the correlation in the storage unit 4 in association with each other (step S6). That is, the determining unit 33 causes the pulse wave propagation time, determined using the selected two pieces of waveform data, and the correlation in shape between the selected two pieces of waveform data to be stored in the storage unit 4 as a pair (a pulse wave propagation time, a correlation) mutually associated.

A plurality of pairs (pulse wave propagation times, correlations) is determined through repetition of steps S4 to S6 described above and is stored in the storage unit 4. When the processing unit 3 decides in step S7 that the determination of a predetermined number of pairs (pulse wave propagation times, correlations) is not completed, the flow returns to step S4, and steps S4 to S6 are performed again. On the other hand, when the processing unit 3 decides in step S7 that the determination of the predetermined number of pairs (pulse wave propagation times, correlations) is completed, the flow proceeds to step S8.

In the present embodiment, specifically, in step S4, the determining unit 33 first selects the pulse-wave data f₁ and f₂ from among the pulse-wave data f₁ of the first portion P1, the pulse-wave data f₂ of the second portion P2, and the pulse-wave data f₃ of the third portion P3 (see FIG. 3).

Next, in step S5, the determining unit 33 determines a difference between the attribute values in the selected two pieces of pulse-wave data f₁ and f₂, that is, a pulse wave propagation time PTT₁₂ between the first portion P1 and the second portion P2 and a correlation in shape between the pulse-wave data f₁ and the pulse-wave data f₂.

In this case, the pulse wave propagation time PTT₁₂ between the first portion P1 and the second portion P2 is a time taken for a pulse wave to propagate from the first portion P1 to the second portion P2. Thus, the pulse wave propagation time PTT₁₂ between the first portion P1 and the second portion P2 is a time-point difference (a time) between a peak of the pulse-wave data f₁ corresponding to one pulse wave and a peak of the pulse-wave data f₂, as shown in FIG. 4.

In the present embodiment, specifically, the determining unit 33 determines the pulse wave propagation time PTT₁₂ between the first portion P1 and the second portion P2 and the correlation in shape between the pulse-wave data f₁ and the pulse-wave data f₂ in a manner described below.

First, the determining unit 33 determines a cross-correlation function R₁₂(τ) for the pulse-wave data f₁ and f₂, the function being given by expression (1) below. Next, the determining unit 33 determines, as the pulse wave propagation time PTT₁₂ between the first portion P1 and the second portion P2, a lag T when the cross-correlation function R₁₂(τ) takes a maximum value R₁₂ₘₐₓ (see FIG. 5). Also, the determining unit 33 determines, as the aforementioned maximum value R₁₂ₘₐₓ, a value representing the correlation in shape between the pulse-wave data f₁ and the pulse-wave data f₂.

The cross-correlation function R₁₂(τ) is a function obtained by normalizing a cross-correlation function r₁₂(τ) for the pulse-wave data f₁ and f₂ so that an autocorrelation reaches 1 for τ=0, the cross-correlation function r₁₂(τ) being given by expression (2) below.

$$\begin{matrix}[ Expression 1 ] & \\ {R}_{12} ( τ ) = \frac{{r}_{12} ( τ )}{\sqrt{{r}_{1} ( 0 ) {r}_{2} ( 0 )}} = \frac{E [ {f}_{1} ( t ) {f}_{2} ( t - τ ) ]}{\sqrt{{E [ {f}_{1} ( t ) )}^{2} ] E [ {( {f}_{2} ( t ) )}^{2} ]}} & ( 1 )\end{matrix}$$

$$\begin{matrix}[ Expression 2 ] & \\ {r}_{12} ( τ ) = E [ {f}_{1} ( t ) {f}_{2} ( t - τ ) ] & ( 2 )\end{matrix}$$

In expressions (1) and (2) noted above,

E[X]: an expected value of X;

r₁(τ): an autocorrelation function for the pulse-wave data f₁ (a cross-correlation function of pulse-wave data f₁ with copy of pulse-wave data f₁ itself);
r₂(τ): an autocorrelation function for the pulse-wave data f₂ (a cross-correlation function of pulse-wave data f₂ with copy of pulse-wave data f₂ itself);
r₁(0): a value of the autocorrelation function r₁(τ), for τ=0;
r₂(0): a value of the autocorrelation function r₂(τ), for τ=0.

Next, in step S6, the determining unit 33 causes the pulse wave propagation time (the lag t when the cross-correlation function R₁₂(τ) takes the maximum value R₁₂ₘₐₓ) PTT₁₂ between the first portion P1 and the second portion P2 and the correlation (the maximum value R₁₂ₘₐₓ) in shape between the pulse-wave data f₁ and the pulse-wave data f₂ to be stored in the storage unit 4 in association with each other.

Next, in step S7, the processing unit 3 decides whether or not the determination of pulse wave propagation times and correlations is completed. Since only the pulse wave propagation time PTT₁₂ between the first portion P1 and the second portion P2 and the correlation R₁₂ₘₐₓ in shape between the pulse-wave data f₁ and the pulse-wave data f₂ have been completed at this point time, the decision in step S7 indicates “No”, and the flow returns to step S4.

Next, in step S4 when it is performed for the second time, the determining unit 33 selects, for example, the pulse-wave data f₁ and f₃ from among the pulse-wave data f₁ of the first portion P1, the pulse-wave data f₂ of the second portion P2, and the pulse-wave data f₃ of the third portion P3 (see FIG. 3).

Next, in step S5 when it is performed for the second time, a pulse wave propagation time PTT₁₃ between the first portion P1 and the third portion P3 and a correlation in shape between the pulse-wave data f₁ and the pulse-wave data f₃ are determined in substantially the same manner as in step S5 performed for the first time described above. First, the determining unit 33 determines a cross-correlation function R₁₃(τ) for the pulse-wave data f₁ and f₃. Next, the determining unit 33 determines, as the pulse wave propagation time PTT₁₃ between the first portion P1 and the third portion P3, the lag T when the cross-correlation function R₁₃(τ) takes a maximum value R₁₃ₘₐₓ. Also, the determining unit 33 determines the maximum value R₁₃ₘₐₓ as a value representing the correlation in shape between the pulse-wave data f₁ and the pulse-wave data f₃.

The cross-correlation function R₁₃(τ) is an expression obtained by normalizing a cross-correlation function r₁₃(τ) for the pulse-wave data f₁ and f₃ so that an autocorrelation reaches 1 for τ=0.

Next, in step S6 when it is performed for the second time, the determining unit 33 causes the pulse wave propagation time (the lag T when the cross-correlation function R₁₃(τ) takes a maximum value R₁₃ₘₐₓ) PTT₁₃ between the first portion P1 and the third portion P3 and the correlation (the maximum value R₁₃ₘₐₓ) in shape between the pulse-wave data f₁ and the pulse-wave data f₃ to be stored in the storage unit 4 in association with each other.

Next, in step S7 when it is performed for the second time, the processing unit 3 decides whether or not the determination of pulse wave propagation times and correlations is completed. At this point in time, since the determination of a pulse wave propagation time between the second portion P2 and the third portion P3 and a correlation in shape between the pulse-wave data f₂ and the pulse-wave data f₃ is not completed, the decision in step S7 when it is performed for the second time indicates “No”, and the flow returns to step S4.

Next, in step S4 when it is performed for the third time, the determining unit 33 selects, for example, the pulse-wave data f₂ and the pulse-wave data f₃ from among the pulse-wave data f₁ of the first portion P1, the pulse-wave data f₂ of the second portion P2, and the pulse-wave data f₃ of the third portion P3 (see FIG. 3).

Next, in step S5 when it is performed for the third time, a pulse wave propagation time PTT₂₃ between the second portion P2 and the third portion P3 and the correlation in shape between the pulse-wave data f₂ and the pulse-wave data f₃ are determined in substantially the same manner as in step S5 performed for the first time described above. First, the determining unit 33 determines a cross-correlation function R₂₃(τ) for the pulse-wave data f₂ and the pulse-wave data f₃. Next, the determining unit 33 determines, as the pulse wave propagation time PTT₂₃ between the second portion P2 and the third portion P3, the lag τ when the cross-correlation function R₂₃(τ) takes a maximum value R₂₃ₘₐₓ. Also, the determining unit 33 determines the maximum value R₂₃ₘₐₓ as a value representing the correlation in shape between the pulse-wave data f₂ and the pulse-wave data f₃.

The cross-correlation function R₂₃(τ) is a function obtained by normalizing a cross-correlation function r₂₃(τ) for the pulse-wave data f₂ and the pulse-wave data f₃ so that an autocorrelation reaches 1 for τ=0.

Next, in step S6 when it is performed for the third time, the determining unit 33 causes the pulse wave propagation time (the lag τ when the cross-correlation function R₂₃(τ) takes the maximum value R₂₃ₘₐₓ) PTT₂₃ between the second portion P2 and the third portion P3 and the correlation (the maximum value R₂₃ₘₐₓ) in shape between the pulse-wave data f₂ and the pulse-wave data f₃ to be stored in the storage unit 4 in association with each other.

Next, in step S7 when it is performed for the third time, the processing unit 3 decides whether or not the determination of pulse wave propagation times and correlations is completed. Since the determination of all the pulse wave propagation times and the correlations has been finished at the time of step S7 performed for the third time, the decision indicates “Yes”, and the flow proceeds to step S8.

As a result of repeating steps S4 to S7 a plurality of times, as described above, the determining unit 33 determines a plurality of pairs (pulse wave propagation times, correlations), and the determined pairs (the pulse wave propagation times, the correlations) are stored in the storage unit 4.

Next, in step S8, the correcting unit 34 corrects the pulse wave propagation times. Specifically, first, the correcting unit 34 reads the pairs (the pulse wave propagation times, the correlations) from the storage unit 4. Next, the correcting unit 34 corrects each pulse wave propagation time (the attribute value difference), based on the correlation paired with the pulse wave propagation time. Specifically, in the present embodiment, the correcting unit 34 corrects the pulse wave propagation time PTT₁₂, based on the correlation (the maximum value R₁₂ₘₐₓ) paired with the pulse wave propagation time PTT₁₂. The correcting unit 34 corrects the pulse wave propagation time PTT₁₃, based on the correlation (the maximum value R₁₃ₘₐₓ) paired with the pulse wave propagation time PTT₁₃. The correcting unit 34 corrects the pulse wave propagation time PTT₂₃, based on the correlation (the maximum value R₂₃ₘₐₓ) paired with the pulse wave propagation time PTT₂₃.

More specifically, the correcting unit 34 corrects the pulse wave propagation time so that the amount of correction on the pulse wave propagation time becomes large as the correlation paired therewith becomes low. For example, assume a case in which the correlation (the maximum value R₁₂ₘₐₓ) in shape between the pulse-wave data f₁ and the pulse-wave data f₂, the correlation (the maximum value R₁₃ₘₐₓ) in shape between the pulse-wave data f₁ and the pulse-wave data f₃, and the correlation (the maximum value R₂₃ₘₐₓ) in shape between the pulse-wave data f₂ and the pulse-wave data f₃ are given by R₁₂ₘₐₓ>R₁₃ₘₐₓ>R₂₃ₘₐₓ. In this case, each of the pulse wave propagation times PTT₁₂, PTT₁₃, and PTT₂₃ is corrected so that the amount of correction on the pulse wave propagation time PTT₂₃ corresponding to the lowest R₂₃ₘₐₓ becomes the largest, and the amount of correction on the pulse wave propagation time PTT₁₂ corresponding to the highest R₁₂ₘₐₓ becomes the smallest.

In the present embodiment, of a pair (the pulse wave propagation time PTT₁₂, the correlation R₁₂ₘₐₓ), a pair (the pulse wave propagation time PTT₁₃, the correlation R₁₃ₘₐₓ), and a pair (the pulse wave propagation time PTT₂₃, the correlation R₂₃ₘₐₓ), the pulse wave propagation time PTT₁₃ in the pair (the pulse wave propagation time PTT₁₃, the correlation R₁₃ₘₐₓ) corresponds to the sum of the pulse wave propagation time PTT₁₂ in the pair (the pulse wave propagation time PTT₁₂, the correlation R₁₂ₘₐₓ) and the pulse wave propagation time PTT₂₃ in the pair (the pulse wave propagation time PTT₂₃, the correlation R₂₃ₘₐₓ). That is, logically, the pulse wave propagation time PTT₁₃ becomes equal to the sum of the pulse wave propagation times PTT₁₂ and PTT₂₃ (logically, PTT₁₃=PTT₁₂+PTT₂₃ holds).

If PTT₁₃=PTT₁₂+PTT₂₃ holds in actual measurement values, it is not necessary to correct the pulse wave propagation times. However, when PTT₁₃=PTT₁₂+PTT₂₃ does not hold in actual measurement values, it can be thought that at least one of the pulse wave propagation times PTT₁₃, PTT₁₂, and PTT₂₃ includes measurement error. Thus, it is necessary to correct at least one of the pulse wave propagation times PTT₁₃, PTT₁₂, and PTT₂₃.

In the present embodiment, based on the magnitude of the correlations R₁₂ₘₐₓ, R₁₃ₘₐₓ, and R₂₃ₘₐₓ, the correcting unit 34 corrects the pulse wave propagation times PTT₁₃, PTT₁₂, and PTT₂₃ so that the difference (|PTT₁₃−(PTT₁₂+PTT₂₃)|) between the pulse wave propagation time PTT₁₃ and the sum of the pulse wave propagation times PTT₁₂ and PTT₂₃ becomes small.

A specific correction method for the pulse wave propagation times is not particularly limited. The correction of the pulse wave propagation times can be performed, for example, based on expression (3) below.

$$\begin{matrix}[ Expression 3 ] & \\ {PTT}_{ij calc} \to \frac{α {PTT}_{ij calc} {R}_{ij max}^{Y} + {\sum }_{n \neq i , j} ( {PTT}_{in calc} + {PTT}_{nj calc} ) {R}_{in max}^{Y} {R}_{nj max}^{Y}}{α {R}_{ij max}^{Y} + {\sum }_{n \neq i , j} {R}_{in max}^{Y} {R}_{nj max}^{Y}} & ( 3 )\end{matrix}$$

In expression (3) noted above,

PTT_{ijcalc}: a pulse wave propagation time between an ith portion and a jth portion, the pulse wave propagation time being determined by the determining unit 33; PTT_{incalc}: a pulse wave propagation time between the ith portion and an nth portion, the pulse wave propagation time being determined by the determining unit 33;

PTT_{njcalc}: a pulse wave propagation time between the nth portion and the jth portion, the pulse wave propagation time being determined by the determining unit 33;

α: a parameter regarding the magnitude of the amount of correction;

Rᵢⱼₘₐₓ: a correlation (a maximum value of a cross-correlation function Rᵢⱼ) in shape between pulse-wave data fᵢ of the ith portion and pulse-wave data fⱼ of the jth portion;

Rᵢₙₘₐₓ: a correlation (a maximum value of a cross-correlation function Rᵢₙ) in shape between the pulse-wave data fᵢ of the ith portion and pulse-wave data fₙ of the nth portion;

Rₙⱼₘₐₓ: a correlation (a maximum value of a cross-correlation function Rₙⱼ) in shape between the pulse-wave data fₙ of the nth portion and the pulse-wave data fⱼ of the jth portion; and

γ: a parameter representing the magnitude of contribution of the correlation with respect to the amount of correction.

The correcting unit 34 may perform the correction of the pulse wave propagation times based on expression (3), noted above, only once or may repeat the correction of the pulse wave propagation times based on expression (3), noted above, a plurality of times so as to gradually bring the difference between the pulse wave propagation time PTT₁₃ and the sum of the pulse wave propagation times PTT₁₂ and PTT₂₃ close to each other. Repeatedly correcting the pulse wave propagation times can enhance the measurement accuracy of the pulse wave propagation times.

Step S9 is performed subsequent to step S8. In step S9, the correcting unit 34 causes the corrected pulse wave propagation times to be displayed on the display unit 5 and also stores the corrected pulse wave propagation times in the storage unit 4. For example, when the measurement apparatus 1 is provided with an output unit, such as a printer, the measurement apparatus 1 may be configured so that the corrected pulse wave propagation times are automatically or manually output after the correcting unit 34 finishes the correction of the pulse wave propagation times.

As described above, in the measurement apparatus 1 according to the first embodiment, each pulse wave propagation time, which is an attribute value difference, is corrected based on a correlation paired with the pulse wave propagation time. In this case, it can be though that a pulse wave propagation speed determined using pulse-wave data in which the correlation in shape is higher has a smaller amount of error. Thus, correcting each pulse wave propagation time based on a correlation paired with the pulse wave propagation time, as in the present embodiment, makes it possible to perform correction having higher accuracy. Accordingly, the pulse wave propagation times can be measured with high accuracy.

Also, when the maximum value of the cross-correlation function for two pieces of waveform data is regarded as a correlation in shape between the two pieces of waveform data, the correlation in shape between the two pieces of waveform data can be evaluated with high accuracy. Accordingly, the pulse wave propagation times can be measured with higher accuracy.

In addition, in the present embodiment, pairs (pulse wave propagations times, correlations) are selected so that a sum of at least one pulse wave propagation time in the selected pairs (the pulse wave propagation times, the correlations) and a sum of the pulse wave propagation times in the remaining pairs (the pulse wave propagation times, the correlations) are comparable with each other, and the pulse wave propagation times are repeatedly corrected so that the difference between the sum of at least one pulse wave propagation time in the selected pairs (the pulse wave propagation times, the correlations) and the sum of the pulse wave propagation times in the remaining pairs (the pulse wave propagation times, the correlation) decreases. Accordingly, the pulse wave propagation times can be measured with higher accuracy.

In the present embodiment, a plurality of pieces of waveform data is generated from one image (one moving image). Thus, for example, it is possible to generate a plurality of pieces of waveform data by a single round of image-capturing, without having to mount detectors on respective portions of a person to be measured. Hence, it is possible to easily generate a plurality of pieces of waveform data, so that the pulse wave propagation times can be measured easily. Also, when a plurality of pieces of waveform data is obtained by capturing one image, the number of pieces of waveform data that is obtained can be easily increased, unlike a case in which detectors are mounted on respective portions of a person to be measured, and waveform data of the respective portions are obtained. That is, it is possible to easily acquire a large amount of waveform data. Determining pulse wave propagation times by using a larger amount of waveform data makes it possible to enhance the measurement accuracy of the pulse wave propagation times. Accordingly, generating a plurality of pieces of waveform data from one image allows pulse wave propagation times to be measured with higher accuracy.

Furthermore, an image can be captured in a contactless manner. Thus, when the waveform data is adapted to be generated from an image, it is possible to easily measure pulse wave propagation times without mounting detectors or the like on a person to be measured.

Other preferable examples in which the present invention is implemented will be described below. In the following description, members having substantially the same functions as those in the first embodiment are denoted by the same numerals, and descriptions thereof are not given hereinafter. In descriptions of a second embodiment and a third embodiment, a reference is also made to FIGS. 1 to 3.

### Second Embodiment

In the first embodiment above, an example in which pieces of pulse-wave data for the respective first portion P1, second portion P2, and third portion P3 are obtained and the determination and the correction of the pulse wave propagation times are performed using the three pieces of pulse-wave data has been described above as one example. However, the present invention is not limited to this.

In the present embodiment, steps S4 to S6 are performed twice.

In step S4 when it is performed for the first time, the waveform-data generating unit 32 selects two pieces of pulse-wave data f₁ and f₂. Next, in step S5 when it is performed for the first time, the determining unit 33 determines a difference between the attribute values in the selected two pieces of pulse-wave data f₁ and f₂, that is, the pulse wave propagation time PTT₁₂ between the first portion P1 and the second portion P2 and the correlation R₁₂ₘₐₓ in shape between the pulse-wave data f₁ and the pulse-wave data f₂.

In step S4 when it is performed for the second time, the waveform-data generating unit 32 selects two pieces of pulse-wave data f₂ and f₃. Next, in step S5 when it is performed for the second time, the determining unit 33 determines a difference between the attribute values in the selected two pieces of pulse-wave data f₂ and f₃, that is, the pulse wave propagation time PTT₂₃ between the second portion P2 and the third portion P3, and the correlation R₂₃ₘₐₓ in shape between the pulse-wave data f₂ and the pulse-wave data f₃.

In step S8, the correcting unit 34 corrects the pulse wave propagation times PTT₁₂ and PTT₂₃, based on the correlations R₁₂ₘₐₓ and R₂₃ₘₐₓ. Specifically, the correcting unit 34 corrects the pulse wave propagation times PTT₁₂ and PTT₂₃ so that, of the pulse wave propagation times PTT₁₂ and PTT₂₃, the amount of correction on the pulse wave propagation time for which the correlation is lower becomes large, and the amount of correction on the pulse wave propagation time for which the correlation is higher becomes small.

For example, the correcting unit 34 may determine a distance between the first portion P1 and the second portion P2 and a distance between the second portion P2 and the third portion P3 and determine pulse wave propagation speeds by using the respective pulse wave propagation times PTT₁₂ and PTT₂₃ by using those distances. Since the pulse wave propagation speeds are directly comparable with each other, the pulse wave propagation speeds may be corrected based on the correlations R₁₂ₘₐₓ and R₂₃ₘₐₓ, and then the pulse wave propagation times may be determined using the corrected pulse wave propagation speeds and the distances. Doing so makes it possible to determine the pulse wave propagation times with higher accuracy.

As in the present embodiment, the correction of the pulse wave propagation times may be performed based on two pairs (pulse wave propagation times, correlations). Even in this case, since the pulse wave propagation times are determined based on the correlations, the pulse wave propagation times can be determined with high accuracy.

### Third Embodiment

A measurement apparatus according to a third embodiment has a configuration that is substantially the same as the measurement apparatus 1 according to the first embodiment, except for the operations of the correcting unit 34. In the present embodiment, in step S8 shown in FIG. 2, the correcting unit 34 selects some of a plurality of pairs (pulse wave propagation times, correlations) determined by the determining unit 33. The correcting unit 34 corrects the pulse wave propagation times (attribute value differences) in the selected pairs (the pulse wave propagation times, the correlations), based on the correlations paired with the pulse wave propagation times.

Specifically, the correcting unit 34 first reads a threshold stored in the storage unit 4. This threshold is a threshold of a correlation regarding the shape of waveform data. Next, the correcting unit 34 excludes the pairs (the pulse wave propagation times, the correlations) in which the correlations are lower than the threshold from the pairs (the pulse wave propagation times, the correlations) to select the pairs (the pulse wave propagation times, the correlations) in which the correlations are higher than or equal to the threshold. The correcting unit 34 corrects each pulse wave propagation time (the attribute value difference) in the selected pair (the pulse wave propagation time, the correlation), based on the correlation paired with the pulse wave propagation time.

Specifically, for example, in step S3, the waveform-data generating unit 32 shown in FIG. 1 generates pieces of pulse-wave data f₁(τ), f₂(τ), f₃(τ), f₄(τ), and f₅(τ) respectively for a first portion P1, a second portion P2, a third portion P3, a fourth portion P4, and a fifth portion P5.

Next, by repeatedly performing steps S4 to S6, the determining unit 33 determines a pair (a pulse wave propagation time PTT₁₂, a correlation R₁₂ₘₐₓ), a pair (a pulse wave propagation time PTT₁₃, a correlation R₁₃ₘₐₓ), a pair (a pulse wave propagation time PTT₁₄, a correlation R₁₄ₘₐₓ), a pair (a pulse wave propagation time PTT₁₅, a correlation R₁₅ₘₐₓ), a pair (a pulse wave propagation time PTT₂₃, a correlation R₂₃ₘₐₓ), a pair (a pulse wave propagation time PTT₂₄, a correlation R₂₄ₘₐₓ), a pair (a pulse wave propagation time PTT₂₅, a correlation R₂₅ₘₐₓ), a pair (a pulse wave propagation time PTT₃₄, a correlation R₃₄ₘₐₓ), a pair (a pulse wave propagation time PTT₃₅, a correlation R₃₅ₘₐₓ), and a pair (a pulse wave propagation time PTT₄₅, a correlation R₄₅ₘₐₓ) shown in FIG. 6 and causes the determined pairs to be stored in the storage unit 4.

Next, in step S8, the correcting unit 34 reads the threshold stored in the storage unit 4. Next, the correcting unit 34 excludes the pairs in which the correlations are lower than the threshold from the pair (the pulse wave propagation time PTT₁₂, the correlation R₁₂ₘₐₓ), the pair (the pulse wave propagation time PTT₁₃, the correlation R₁₃ₘₐₓ), the pair (the pulse wave propagation time PTT₁₄, the correlation R₁₄ₘₐₓ), the pair (the pulse wave propagation time PTT₁₅, the correlation R₁₅ₘₐₓ), the pair (the pulse wave propagation time PTT₂₃, the correlation R₂₃ₘₐₓ), the pair (the pulse wave propagation time PTT₂₄, the correlation R₂₄ₘₐₓ), the pair (the pulse wave propagation time PTT₂₅, the correlation R₂₅ₘₐₓ), the pair (the pulse wave propagation time PTT₃₄, the correlation R₃₄ₘₐₓ), the pair (the pulse wave propagation time PTT₃₅, the correlation R₃₅ₘₐₓ), and the pair (the pulse wave propagation time PTT₄₅, the correlation R₄₅ₘₐₓ). The correcting unit 34 selects at least one of the pairs (the pulse wave propagation times, the correlations) that are not excluded. Specifically, the correcting unit 34 selects some pairs (the pulse wave propagation times, the correlations), that is, the pair (the pulse wave propagation time PTT₁₃, the correlation R₁₃ₘₐₓ), the pair (the pulse wave propagation time PTT₁₅, the correlation R₁₅ₘₐₓ), the pair (the pulse wave propagation time PTT₃₄, the correlation R₃₄ₘₐₓ), and the pair (the pulse wave propagation time PTT₄₅, the correlation R₄₅ₘₐₓ). The correcting unit 34 corrects the selected pulse wave propagation times PTT₁₃, PTT₁₅, PTT₃₄, and PTT₄₅, based on the correlation R₁₃ₘₐₓ, R₁₅ₘₐₓ, R₃₄ₘₐₓ, and R₄₅ₘₐₓ paired with the pulse wave propagation times.

A method that is substantially the same as the correction method described in the first embodiment can be employed as a correction method for the pulse wave propagation times; however, in the present embodiment, for example, the pulse wave propagation times can be preferably corrected using expression (4) below.

$${PTT}_{ij calc} \to \frac{α {PTT}_{ij calc} {R}_{ij max}^{′Y} + β {\sum }_{n \neq i , j} ( {PTT}_{in calc} + {PTT}_{nj calc} ) {R}_{in max}^{′Y} {R}_{nj max}^{′Y} + {\sum }_{m , n \neq i , j} ( {PTT}_{in calc} + {PTT}_{mn calc} + {PTT}_{nj calc} ) {R}_{in max}^{′Y} {R}_{mn max}^{′Y} {R}_{nj max}^{′Y}}{α {R}_{ij max}^{′Y} + β {\sum }_{n \neq i , j} {R}_{in max}^{′Y} {R}_{nj max}^{′Y} + {\sum }_{m , n \neq i , j} {R}_{in max}^{′Y} {R}_{mn max}^{′Y} {R}_{nj max}^{′Y}}$$

$${R}_{ij max}^{′} = \{ \begin{matrix}{R}_{ij max} & ( {R}_{ij max} \geq {R}_{s} ) \\ 0 & ( {R}_{ij max} < {R}_{i} )\end{matrix}$$

$${R}_{mn max}^{′} = \{ \begin{matrix}{R}_{mn max} & ( {R}_{mn max} \geq {R}_{s} ) \\ 0 & ( {R}_{mn max} < {R}_{i} )\end{matrix}$$

$${R}_{nj max}^{′} = \{ \begin{matrix}{R}_{nj max} & ( {R}_{nj max} \geq {R}_{s} ) \\ 0 & ( {R}_{nj max} < {R}_{i} )\end{matrix}$$

In expression (4) noted above,

PTT_{ijcalc}: a pulse wave propagation time between an ith portion and a jth portion, the pulse wave propagation time being determined by the determining unit 33;

PTT_{incalc}: a pulse wave propagation time between the ith portion and an nth portion, the pulse wave propagation time being determined by the determining unit 33;

PTT_{njcalc}: a pulse wave propagation time between the nth portion and the jth portion, the pulse wave propagation time being determined by the determining unit 33;

PTT_{nmcalc}: a pulse wave propagation time between the nth portion and an mth portion, the pulse wave propagation time being determined by the determining unit 33;

PTT_{mjcalc}: a pulse wave propagation time between the mth portion and the jth portion, the pulse wave propagation time being determined by the determining unit 33;

Rᵢⱼₘₐₓ: a correlation (a maximum value of a cross-correlation function Rᵢⱼ) in shape between pulse-wave data fᵢ of the ith portion and pulse-wave data fⱼ of the jth portion;

Rᵢₙₘₐₓ: a correlation (a maximum value of a cross-correlation function Rᵢₙ) in shape between the pulse-wave data fᵢ of the ith portion and pulse-wave data fₙ of the nth portion;

Rₙⱼₘₐₓ: a correlation (a maximum value of a cross-correlation function Rₙⱼ) in shape between the pulse-wave data fₙ of the nth portion and the pulse-wave data fⱼ of the jth portion;

Rₙₘₘₐₓ: a correlation (a maximum value of a cross-correlation function Rᵢₙ) in shape between the pulse-wave data fₙ of the nth portion and pulse-wave data fₘ of the mth portion;

Rₙⱼₘₐₓ: a correlation (a maximum value of cross-correlation function Rₙⱼ) in shape between the pulse-wave data fₘ of the mth portion and the pulse-wave data fⱼ of the jth portion;

Rₛ: a threshold of the correlation;

α: a parameter regarding the magnitude of the amount of correction;

β: a parameter representing the magnitude of contribution of the sum of two pulse wave propagation times with respect to the amount of correction; and

γ: a parameter representing the magnitude of contribution of the correlation with respect to the amount of correction.

When some pulse wave propagation times of a plurality of pairs (pulse wave propagation times, correlations) determined by the determining unit 33 are corrected, as in the third embodiment, the pulse wave propagation times can be measured with higher accuracy. Specifically, for example, the pulse wave propagation times can be measured with higher accuracy by selecting some pairs (pulse wave propagation times, correlations) in which the correlations are high from among the plurality of pairs (the pulse wave propagation times, the correlations) determined by the determining unit 33 and correcting the pulse wave propagation times in the selected pairs (the pulse wave propagation times, the correlations).

### Fourth Embodiment

In the first to third embodiments, the description has been given of examples in which a plurality of pieces of pulse-wave data is generated from a moving image captured by the image-capture unit 2. However, the present invention is not limited to this.

For example, the measurement apparatus may be a measurement apparatus that does not comprise an image-capture unit, that obtains an image (a moving image) from a directly or wirelessly connected external device, and that generates a plurality of pieces of pulse-wave data from the obtained image data. In this case, it is preferable that the measurement apparatus comprise an obtaining unit that obtains, from the external device, information for generating pulse-wave data.

The information for generating a plurality of pieces of pulse-wave data is not limited to an image, such as a moving image. For example, the measurement apparatus may comprise a detector for directly obtaining a waveform. Specific examples of the detector include, for example, a pressure sensor for detecting a pressure change, an optical sensor for detecting a light intensity, a microphone for collecting sound, an ultrasonic sensor for detecting an ultrasonic wave, a displacement sensor, such as a seismometer, for detecting a displacement, and so on. The measurement apparatus may comprise a plurality of detectors for detecting pulse waves at portions where they are installed respectively. When the measurement apparatus has a detecting unit for directly detecting a pulse wave or is a measurement apparatus to which a pulse wave is directly input from a detector or the like, the measurement apparatus does not necessarily have to be provided with the image-capture unit 2, the obtaining unit 31, and the waveform-data generating unit 32.

(Modification)

One example of a modification of the above-described embodiments will be described below.

In the above embodiments, the description has been given of examples in which the pulse wave propagation times and the correlations are determined using cross-correlation functions. However, in the present invention, a determination method for the attribute value differences and the corrections is not particularly limited. For example, the attribute value differences and the correlations may also be determined using a cross-covariance function given in expression (5) below.

$$\begin{matrix}[ Expression 5 ] & \\ {C}_{ij} ( τ ) = \frac{{C}_{ij} ( τ )}{\sqrt{{V}_{i} {V}_{j}}} = \frac{E [ ( {f}_{i} ( t ) - {μ}_{i} ) ( {f}_{j} ( τ - t ) - {μ}_{j} ) ]}{\sqrt{E [ {( {f}_{i} ( t ) - {μ}_{i} )}^{2} ] {E ( {f}_{j} ( t ) - {μ}_{j} )}^{2} ]}} & ( 5 )\end{matrix}$$

In expression (5) noted above,

cᵢⱼ(τ): a cross-covariance function for the pulse-wave data fᵢ of the ith portion and the pulse-wave data fⱼ of the jth portion;

Cᵢⱼ(τ): a function obtained by normalizing the cross-covariance function cᵢⱼ(τ) so that an autocovariance reaches 1 for the lag τ=0;

V: a distribution; and

μ: an average

The pulse wave propagation time between the ith portion and the jth portion is given as the lag T when the cross-covariance function Cᵢⱼ(τ) takes a maximum value, and a maximum value Cᵢⱼₘₐₓ of the cross-covariance function Cᵢⱼ(τ) is given as a correlation between the pulse-wave data fᵢ of the ith portion and the pulse-wave data fⱼ of the jth portion.

In the third embodiment, the description has been given of an example in which pairs (pulse wave propagation times, correlations) in which the correlations are higher than the threshold are selected, and the pulse wave propagation times in the selected pairs (the pulse wave propagation time, the correlations) are corrected based on the correlations. However, in the present invention, a method for selecting some pairs (the pulse wave propagation times, the correlations) from among the plurality of pairs (the pulse wave propagation times, the correlations) is not particularly limited.

For example, pairs (pulse wave propagation times, correlations) determined using pulse-wave data whose quality satisfies a set reference (a predetermined quality) may be selected. Herein, the set reference for the quality may be set as appropriate, for example, based on a parameter, such as an amplitude of the pulse-wave data, the data length of the pulse-wave data, or an S/N ratio of the pulse-wave data.

The description has been given of examples in which, in the embodiments above, the maximum value Rₘₐₓ is used as a correlation in shape between two pieces of waveform data, and in the modification above, the maximum value Cᵢⱼₘₐₓ is used as a correlation in shape between two pieces of waveform data. However, in the present invention, the correlation in shape between two pieces of waveform data is not limited to the above-described maximum value. For example, the correlation in shape between two pieces of waveform data may be an index indicating a mean squared error between two pieces of waveform data or a degree of deviation in shape between two pieces of waveform data, such as a Euclidean distance. In this case, it is preferable that the amount of correction on the attribute value difference be increased as the degree of deviation increases, and the amount of correction on the attribute value difference be reduced as the degree of deviation decreases.

In the embodiments above, the description has been given of examples in which the waveform data is pulse-wave data, which is waveform data indicating changes over time in an observation value at an arbitrary spot. That is, the description has been given of examples in which the attribute values are time points. However, in the present invention, the waveform data is not limited to waveform data in which the attribute values are time points.

In the present invention, the waveform data may be, for example, waveform data in which the attribute values are positions. Specifically, the waveform data may be, for example, waveform data indicating spatial distribution of observation values at an arbitrary time point.

In the present invention, for example, the attribute value difference may be determined as a phase difference.

In the above embodiment, the description has been given of examples in which the pulse wave propagation times and the correlations are stored in the storage unit 4 in association with each other. However, in the present invention, the attribute value differences and the correlations do not necessarily have to be stored in association with each other. For example, the attribute value differences and the correlations may each be stored separately in association with an ID, such as a serial number, the name of a measurement portion, or the like.

In the embodiments above, the description has been given of examples in which the measurement apparatus has a display unit. However, the present invention is not limited to this configuration. For example, the measurement apparatus according to the present invention does not have to comprise a display unit.

1. A measurement apparatus comprising:
a determining unit that repeats selecting two pieces of waveform data from among three or more pieces of waveform data including attribute values and observation values corresponding to the attribute values and determining a difference between the attribute values in the selected two pieces of waveform data and a correlation in shape between the selected two pieces of waveform data, to determine difference-correlation pairs, each being a pair of the attribute value difference and the correlation; and
a correcting unit that corrects the attribute value difference, based on the correlation paired with the attribute value difference.

2. The measurement apparatus according to claim 1,
wherein the determining unit determines, as the correlation, a maximum value of a cross-correlation function or a cross-covariance function for the selected two pieces of waveform data.

3. The measurement apparatus according to claim 1,
wherein the correcting unit corrects the attribute value difference so that an amount of correction on the attribute value difference increases as the correlation paired therewith decreases.

4. The measurement apparatus according to claim 1,
wherein the determining unit determines three or more difference-correlation pairs.

5. The measurement apparatus according to claim 1,
wherein the correcting unit selects all or some of the difference-correlation pairs determined by the determining unit and corrects the attribute value difference of the selected difference-correlation pairs, based on the correlation paired with the attribute value difference.

6. The measurement apparatus according to claim 5,
wherein the correcting unit selects all or some of the difference-correlation pairs so that a sum of the attribute value differences in at least one of the selected difference-correlation pairs and a sum of the attribute value differences in the remaining difference-correlation pairs are comparable with each other and corrects the attribute value differences so that the difference between the sum of the attribute value differences in the at least one of the selected difference-correlation pairs and the sum of the attribute value differences in the remaining difference-correlation pairs decreases.

7. The measurement apparatus according to claim 6,
wherein the correcting unit repeatedly performs correction for reducing the difference between the sum of the attribute value differences in the at least one of the selected difference-correlation pairs and the sum of the attribute value differences in the remaining difference-correlation pairs.

8. The measurement apparatus according to claim 5,
wherein the correcting unit selects some difference-correlation pairs in which the correlations are high from among the difference-correlation pairs determined by the determining unit.

9. The measurement apparatus according to claim 1, further comprising:
an obtaining unit that obtains information for generating the waveform data.

10. The measurement apparatus according to claim 9,
wherein the obtaining unit obtains an image as information for generating the waveform data.

11. The measurement apparatus according to claim 10, further comprising:
a waveform-data generating unit that generates a plurality of pieces of the waveform data from one image obtained by the obtaining unit.

12. The measurement apparatus according to claim 11,
wherein the waveform-data generating unit generates a plurality of pieces of the waveform data from one moving image.

13. The measurement apparatus according to claim 1,
wherein the waveform data is waveform data indicating changes over time in an observation value at an arbitrary spot.

14. The measurement apparatus according to claim 1,
wherein the waveform data is waveform data indicating spatial distribution of observation values at an arbitrary time point.

15. The measurement apparatus according to claim 1,
wherein the correcting unit determines the attribute value difference as a phase difference.

16. The measurement apparatus according to claim 13,
wherein the waveform data is pulse-wave data, and
the determining unit determines a pulse wave propagation time as the attribute value difference.

17. A measurement method for a difference between attribute values,
wherein selecting two pieces of waveform data from among three or more pieces of waveform data including attribute values and observation values corresponding to the attribute values and determining a difference between the attribute values in the selected two pieces of waveform data and a correlation in shape between the selected two pieces of waveform data are repeated to determine difference-correlation pairs, each being a pair of the attribute value difference and the correlation; and
the attribute value difference is corrected based on the correlation paired with the attribute value difference.

18. The measurement apparatus according to claim 2,
wherein the correcting unit corrects the attribute value difference so that an amount of correction on the attribute value difference increases as the correlation paired therewith decreases.

19. The measurement apparatus according to claim 2,
wherein the determining unit determines three or more difference-correlation pairs.

20. The measurement apparatus according to claim 3,
wherein the determining unit determines three or more difference-correlation pairs.
